(19) United States
(12) Patent Application Publication
(10) Pub. No.: US 2004/0023935 A1
(43) Pub. Date: Feb. 5, 2004

(54) Inhalation compositions, methods of use thereof, and process for preparation of same

(75) Inventors: Partha S. Banerjee, San Ramon, CA (US); Ramana R. Malladi, Hayward, CA (US); Imtiaz A. Chaudry, Napa, CA (US)

Correspondence Address: STEPHANIE SEIDMAN, HELLER EHRMAN WHITE & MCAULIFFE LLP, 7TH FL., 4350 LA JOLLA VILLAGE DRIVE, SAN DIEGO, CA, 92122-1246, US

(73) Assignee: Dey, L.P.

(21) Appl. No.: 10/212,573

(22) Filed: Aug. 2, 2002

(51) Int. Cl. A61K 31/58; A61K 31/573

(52) U.S. Cl. 514/174; 514/179

(57) ABSTRACT

Bronchodilating concentrates and diluted compositions, methods of use thereof, and processes for making the concentrates and diluted compositions, are provided. The compositions are intended for administration as a nebulized aerosol. Methods for treatment, prevention, or amelioration of one or more symptoms of bronchoconstrictive disorders using the compositions provided herein are also provided.

### FIELD OF THE INVENTION

Inhalation compositions and methods are provided for pulmonary or nasal delivery of a corticosteroid. In particular, the compositions and methods herein include beclomethasone dipropionate (BDP), beclomethasone monopropionate (BMP), flunisolide, triamcinolone acetonide, ciclesonide, mometasone, mometasone furoate (Asmanex® Twisthaler™, Schering-Plough Corporation, Kenilworth, N.J.), RPR 106541, fluticasone and fluticasone propionate, or derivatives thereof. The compositions are propellant-free, sterile unit dose or multidose solutions intended for administration via oral inhalation (nebulization) or nasal spray.

### BACKGROUND OF THE INVENTION

Bronchoconstrictive disorders affect millions worldwide. Such disorders include asthma (including bronchial asthma, allergic asthma and intrinsic asthma, e.g., late asthma and airway hyper-responsiveness), chronic bronchitis and other chronic obstructive pulmonary diseases. Corticosteroids have been developed to treat these conditions. Such corticosteroids include, but are not limited to, steroidal anti-inflammatory agents such as beclomethasone dipropionate (BDP), beclomethasone monopropionate (BMP), flunisolide, triamcinolone acetonide, ciclesonide, mometasone, mometasone furoate (Asmanex® Twisthaler™, Schering-Plough Corporation, Kenilworth, N.J.), RPR 106541, fluticasone and fluticasone propionate.

Delivery of therapeutic compounds directly to affected lung tissues has several advantages. One prime advantage is that high local concentration can be achieved in the lungs while the systemic concentration is kept below that likely to cause adverse side effects. Similar considerations apply for nasal delivery.

Several means have been developed to deliver compounds directly to the passages of the lung or nose. The most common form, especially for water-insoluble drugs, is a powder suspension that is propelled into the mouth while the patient inhales. Propulsion is accomplished by use of a pressurized gas (metered dose inhalers, MDI) or by any of a variety of mechanical means of entraining a fine powder into a gas or air stream (dry powder inhalers, DPI).

The particle size distribution of the aerosolized drug compositions is very important to the therapeutic efficacy of the drug when delivered by inhalation. Additional considerations for the use of powder type drug delivery devices for inhalation include the limited amount of drug that can be contained in one or two puffs from the device and the need for the user to skillfully coordinate hand activation of the device with inhalation. This latter limitation is particularly important for those patients who are disabled, children or elderly.

Nebulizers offer an alternative method of administering therapeutic agents to the lungs of these group of patients. If the drug is not soluble in water, the droplet size of nebulized drug-containing suspensions cannot be smaller than that of the suspended drug particles. Therefore, the finer droplets produced from these systems would not contain any drug. In vitro studies also indicate that suspensions are less efficient than solubilized systems in nebulizing and delivering the drug.

Thus, there is a need to develop improved systems that can solubilize water-insoluble drugs for nebulization. Anti-inflammatory corticosteroids, which are essentially water-insoluble drugs that act on inflammatory cells in the respiratory mucosa, are a type of therapeutic compounds in need of improved inhaled delivery.

### SUMMARY OF THE INVENTION

Compositions and methods for treatment, prevention, or amelioration of one or more symptoms of bronchoconstrictive disorders are provided. The compositions provided herein include a “concentrate” that is stable at ambient temperature. In one embodiment, the composition is an aqueous concentrate that is stable at ambient temperature. This concentrate is diluted prior to administration to a patient. In one embodiment, the “diluted composition” is administered by nebulization. The concentrates and diluted compositions provided herein are propellant-free formulations.

The compositions provided herein include a corticosteroid anti-inflammatory agent. In one embodiment, the compositions provided herein include a corticosteroid anti-inflammatory agent and an alkali or alkaline earth metal halide. In certain embodiments, the alkali or alkaline earth metal halide is present at a concentration of about 0.01% to about 0.89% by weight. Corticosteroid anti-inflammatory agents for use herein include, but are not limited to, beclomethasone dipropionate (BDP), beclomethasone monopropionate (BMP), flunisolide, triamcinolone acetonide, ciclesonide, mometasone, mometasone furoate (Asmanex® Twisthaler™, Schering-Plough Corporation, Kenilworth, N.J.), RPR 106541, fluticasone and fluticasone propionate. In one embodiment, the corticosteroid anti-inflammatory agent is fluticasone or fluticasone propionate.

The compositions provided herein are formulated so as to remain stable over a relatively long period of time. For example, the compositions provided herein are stored between −15° C. and 30° C., or between 2° C. and 8° C. In one embodiment, the compositions are stored at 5° C. In another embodiment, the compositions are stored at 25° C.

In one embodiment, the compositions provided herein, including concentrates and compositions for nebulization, include (i) a corticosteroid anti-inflammatory agent such as beclomethasone dipropionate (BDP), beclomethasone monopropionate (BMP), flunisolide, triamcinolone acetonide, ciclesonide, mometasone, mometasone furoate (Asmanex® Twisthaler™, Schering-Plough Corporation, Kenilworth, N.J.), RPR 106541, fluticasone or fluticasone propionate; (ii) a surfactant; (iii) sodium chloride; and (iv) water. It has been found herein that the presence of sodium chloride at certain concentrations in the compositions provided herein imparts increased stability to the compositions, including the concentrates and diluted compositions.

In certain embodiments, the compositions are administered via nebulization. Administration of a nebulized aerosol is preferred over the use of dry powders for inhalation in certain patient populations, including pediatric and geriatric groups.

Also provided herein are combinations of a concentrate and a diluent. Diluents for use herein include, but are not limited to, aqueous buffer solution, water for injection, isotonic saline solution, saline solution, sterile water, water/alcohol mixtures, etc.

Also provided herein are kits containing a composition provided herein, such as a concentrate or a diluted composition, and a nebulizer. The kits optionally contain instructions for use. Any nebulizer is contemplated for use in the kits and methods provided herein. In particular, the nebulizers for use herein nebulize liquid formulations, including the compositions provided herein, containing no propellant. The nebulizer may produce the nebulized mist by any method known to those of skill in the art, including, but not limited to, compressed air, ultrasonic waves, or vibration. The nebulizer may further have an internal baffle. The internal baffle, together with the housing of the nebulizer, selectively removes large droplets from the mist by impaction and allows the droplets to return to the reservoir. The fine aerosol droplets thus produced are entrained into the lung by the inhaling air/oxygen.

Methods for the treatment, prevention, or amelioration of one or more symptoms of bronchoconstrictive disorders, including, but not limited to, asthma, including, but not limited to, bronchial asthma, allergic asthma and intrinsic asthma, e.g., late asthma and airway hyper-responsiveness; chronic bronchitis; and other chronic obstructive pulmonary diseases are provided. The methods involve administering an effective amount of a pharmaceutical composition provided herein to a patient in need of such treatment.

Articles of manufacture, containing packaging material, a composition provided herein, such as a concentrate or a diluted composition, which is useful for treatment, prevention or amelioration of one or more symptoms of diseases or disorders associated with undesired and/or uncontrolled bronchoconstriction, and a label that indicates that the composition is used for treatment, prevention or amelioration of one or more symptoms of diseases or disorders associated with undesired and/or uncontrolled bronchoconstriction, are also provided.

### DETAILED DESCRIPTION OF PREFERRED EMBODIMENTS

A. Definitions

Unless defined otherwise, all technical and scientific terms used herein have the same meaning as is commonly understood by one of ordinary skill in the art to which this invention belongs. All patents, applications, published applications and other publications are incorporated by reference in their entirety. In the event that there are a plurality of definitions for a term herein, those in this section prevail unless stated otherwise.

As used herein, a “concentrate” is a composition provided herein that must be diluted prior to administration to a subject in need thereof. Concentrates generally are more stable at ambient temperature than diluted compositions.

As used herein, a “diluted composition” is a composition for direct administration to a subject in need thereof. In certain embodiments, a diluted composition is prepared by addition of a concentrate to an aqueous phase.

As used herein, an “aqueous phase” is water or saline. “Aqueous phase” does not contain any corticosteroid until it is mixed with the molten surfactant phase.

As used herein, fluticasone refers to (6α,9α,11α,16α,17α)-6,9-difluoro-11-hydroxy-16-methyl-3-oxo-17-hydroxyandrosta-1,4-diene-17-carbothioic acid, S-fluoromethyl ester. Fluticasone propionate refers to (6α,9α,11α,16α,17α)-6,9-difluro-11-hydroxy-16-methyl-3-oxo-17-(1-oxopropoxy)androsta-1,4-diene-17-carbothioic acid, S-fluoromethyl ester.

As used herein, beclomethasone dipropionate refers to 9-chloro-11_{β},17,21-trihydroxy-16_{β}-methylpregna-1,4-diene-3,20-dione 17,21-dipropionate.

As used herein, beclomethasone monopropionate refers to 9-chloro-11_{β},17,21-trihydroxy-16_{β}-methylpregna-1,4diene-3,20-dione monopropionate.

As used herein, flunisolide refers to 6_{α}-fluoro-11_{β},17,21-tetrahydroxy-pregna-1,4-diene-3,20-dione cyclic-16,17acetal with acetone.

As used herein, triamcinolone acetonide refers to 9-fluoro-11_{β},16_{α},17,21-tetrahydroxypregna-1,4-diene-3,20-dione cyclic 16,17-acetal with acetone.

As used herein, ciclesonide refers to 11_{β},16_{α},17,21-tetrahydroxypregna-1,4-diene-3,20-dione, cyclic 16,17-acetal with cyclohexanecarboxaldehyde.

As used herein, mometasone refers to 9_{α},21-dichloro-11_{β},17-dihydroxy-16_{α}-methylpregna-1,4-diene-3,20-dione.

As used herein, mometasone furoate refers to 9_{α},21-dichloro-11_{β},17-dihydroxy-16_{α}-methylpregna-1,4-diene-3,20-dione 17-(2-furoate).

As used herein, RPR 106541 refers to:

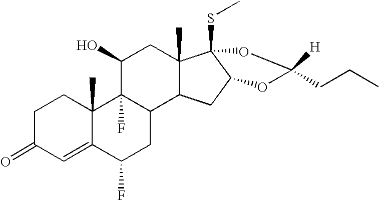

As used herein, an aerosol is liquid or particulate matter dispersed in air. Aerosols include dispersions of liquids, including aqueous and other solutions, and solids, including powders, in air.

As used herein, a nebulized solution refers to a solution that is dispersed in air to form an aerosol. Thus, a nebulized solution is a particular form of an aerosol.

As used herein, a nebulizer is an instrument that is capable of generating very fine liquid droplet for inhalation into the lung. Within this instrument, the nebulizing liquid or solution is atomized into a mist of droplets with a broad size distribution by methods known to those of skill in the art, including, but not limited to, compressed air, ultrasonic waves, or a vibrating orifice. Nebulizers may further contain, e.g., a baffle which, along with the housing of the instrument, selectively removes large droplets from the mist by impaction. Thus, the mist inhaled into the lung contains fine aerosol droplets.

As used herein, a combination refers to two component items, such as compositions or mixtures, that are intended for use either together or sequentially. The combination may be provided as a mixture of the components or as separate components packaged or provided together, such as in a kit.

As used herein, packaging material refers to a physical structure housing components (i.e., a composition, diluent, and/or a nebulizer) of a kit. The packaging material can maintain the components sterilely, and can be made of material and containers commonly used for such purposes (e.g., paper, corrugated fiber, glass, plastic, foil, ampoules, vials, tubes and others). The label or packaging insert can include appropriate written instructions, for example, practicing a method provided herein.

As used herein, the stability of a composition provided herein refers to the length of time at a given temperature that greater than 80%, 85%, 90% or 95% of the initial amount of active ingredient, e.g., fluticasone, is present in the composition. Thus, for example, a composition that is stable for 30 days at 25° C. would have greater than 80%, 85%, 90% or 95% of the initial amount of active ingredient present in the composition at 30 days following storage at 25° C.

As used herein, pharmaceutically acceptable derivatives of a compound include salts, esters, enol ethers, enol esters, acids, bases, solvates, hydrates or prodrugs thereof. Such derivatives may be readily prepared by those of skill in this art using known methods for such derivatization. The compounds produced may be administered to animals or humans without substantial toxic effects and either are pharmaceutically active or are prodrugs. Pharmaceutically acceptable salts include, but are not limited to, amine salts, such as but not limited to N,N′-dibenzylethylenediamine, chloroprocaine, choline, ammonia, diethanolamine and other hydroxyalkylamines, ethylenediamine, N-methylglucamine, procaine, N-benzylphenethylamine, 1-para-chlorobenzyl-2-pyrrolidin-1′-ylmethylbenzimidazole, diethylamine and other alkylamines, piperazine and tris(hydroxymethyl)aminomethane; alkali metal salts, such as but not limited to lithium, potassium and sodium; alkali earth metal salts, such as but not limited to barium, calcium and magnesium; transition metal salts, such as but not limited to zinc; and other metal salts, such as but not limited to sodium hydrogen phosphate and disodium phosphate; and also including, but not limited to, salts of mineral acids, such as but not limited to hydrochlorides and sulfates; and salts of organic acids, such as but not limited to acetates, lactates, malates, tartrates, citrates, ascorbates, succinates, butyrates, valerates and fumarates. Pharmaceutically acceptable esters include, but are not limited to, alkyl, alkenyl, alkynyl, aryl, heteroaryl, aralkyl, heteroaralkyl, cycloalkyl and heterocyclyl esters of acidic groups, including, but not limited to, carboxylic acids, phosphoric acids, phosphinic acids, sulfonic acids, sulfinic acids and boronic acids. Pharmaceutically acceptable enol ethers include, but are not limited to, derivatives of formula C═C(OR) where R is hydrogen, alkyl, alkenyl, alkynyl, aryl, heteroaryl, aralkyl, heteroaralkyl, cycloalkyl ar heterocyclyl. Pharmaceutically acceptable enol esters include, but are not limited to, derivatives of formula C═C(OC(O)R) where R is hydrogen, alkyl, alkenyl, alkynyl, aryl, heteroaryl, aralkyl, heteroaralkyl, cycloalkyl ar heterocyclyl. Pharmaceutically acceptable solvates and hydrates are complexes of a compound with one or more solvent or water molecule, preferably 1 to about 100, more preferably 1 to about 10, most preferably one to about 2, 3 or 4, solvent or water molecules.

As used herein, treatment means any manner in which one or more of the symptoms of a condition, disorder or disease are ameliorated or otherwise beneficially altered. Treatment also encompasses any pharmaceutical use of the compositions herein, such as use for treating cancer.

As used herein, amelioration of the, symptoms of a particular disorder by administration of a particular pharmaceutical composition refers to any lessening, whether permanent or temporary, lasting or transient that can be attributed to or associated with administration of the composition.

As used herein, a prodrug is a compound that, upon in vivo administration, is metabolized or otherwise converted to the biologically, pharmaceutically or therapeutically active form of the compound. To produce a prodrug, the pharmaceutically active compound is modified such that the active compound will be regenerated by metabolic processes. The prodrug may be designed to alter the metabolic stability or the transport characteristics of a drug, to mask side effects or toxicity, to improve the flavor of a drug or to alter other characteristics or properties of a drug. By virtue of knowledge of pharmacodynamic processes and drug metabolism in vivo, those of skill in this art, once a pharmaceutically active compound is known, can design prodrugs of the compound (see, e.g., Nogrady (1985) Medicinal Chemistry A Biochemical Approach, Oxford University Press, New York, pages 388-392).

It is to be understood that the compounds for use in the compositions and methods provided herein may contain chiral centers. Such chiral centers may be of either the (R) or (S) configuration, or may be a mixture thereof. Thus, the compounds for use in the compositions provided herein may be enantiomerically pure, or be stereoisomeric or diastereomeric mixtures. It is to be understood that the chiral centers of the compounds provided herein may undergo epimerization in vivo. Thus, one of skill in the art will recognize that administration of a compound in its (R) form is equivalent, for compounds that undergo epimerization in vivo, to administration of the compound in its (S) form.

B. Compositions for Use in Treatment, Prevention, or Amelioration of One or More Symptoms of Bronchoconstrictive Disorders

Compositions, including concentrates and diluted compositions, containing (i) a corticosteroid, such as beclomethasone dipropionate (BDP), beclomethasone monopropionate (BMP), flunisolide, triamcinolone acetonide, ciclesonide, mometasone, mometasone furoate, RPR 106541, fluticasone or fluticasone propionate; (ii) a surfactant; (iii) sodium chloride; and (iv) water are provided. In one embodiment, the compositions are concentrates, which are diluted prior to administration to a subject in need thereof. In another embodiment, the compositions are diluted compositions which are directly administered to a subject in need thereof.

In one embodiment, the concentrates provided herein contain about 1% to about 95%, or about 15% to about 85%, or about 30% to about 80%, water. In another embodiment, the diluted compositions provided herein contain about 5% to about 99%, or about 60% to about 99%, or about 80% to about 99%, water. In another embodiment, the diluted compositions provided herein contain about 40% to about 90% water. In other embodiments, the compositions provided herein contain at most about 69% by weight water.

The diluted compositions are propellant-free compositions for administration via nebulization. The compositions are sterile filtered and filled in vials, including unit dose vials providing sterile unit dose formulations which are used in a nebulizer and suitably nebulized. Each unit dose vial is sterile and is suitably nebulized without contaminating other vials or the next dose. The bulk sterile formulation is sterilized by steam, gamma radiation, filtration, or is prepared using sterile steroidal powder.

The unit dose vials are formed in a form-fill-seal machine or by any other suitable method known to those of skill in the art. The vials may be made of plastic materials that are suitably used in these processes. For example, plastic materials for preparing the unit dose vials include, but are not limited to, low density polyethylene, high density polyethylene, polypropylene and polyesters. In one embodiment, the plastic material is low density polyethylene.

Aqueous compositions containing corticosteroids, including beclomethasone dipropionate, budesonide and triamcinolone acetonide, for nasal and pulmonary delivery are disclosed in U.S. Pat. No. 6,241,969. This patent discloses an anhydrous “concentrate” that must be maintained at elevated temperature. The “concentrate” of U.S. Pat. No. 6.241,969 is diluted with aqueous phase at elevated temperature to form a solution which may be stored at ambient temperature prior to administration by nebulization. U.S. Pat. No. 6,241,969 does not disclose any “concentrates” that contain water or sodium chloride.

1. Beclomethasone Dipropionate (BDP), Beclomethasone Monopropionate (BMP), Flunisolide, Triamcinolone Acetonide, Ciclesonide, Mometasone, Mometasone Furoate, RPR 106541, Fluticasone or Fluticasone Propionate

Beclomethasone dipropionate (BDP) is 9-chloro-11_{β},17,21-trihydroxy-16_{β}-methylpregna-1,4-diene-3,20-dione 17,21-dipropionate. Beclomethasone monopropionate (BMP) is 9-chloro-11_{β},17,21-trihydroxy-16_{β}-methylpregna-1,4-diene-3,20-dione monopropionate. Flunisolide is 6_{α}-fluoro-11_{β},16_{α},17,21-tetrahydroxy-pregna-1,4-diene-3,20-dione cyclic-16,17-acetal with acetone. Triamcinolone acetonide is 9-fluoro-11_{β},16_{α},17,21-tetrahydroxypregna-1,4-diene-3,20-dione cyclic 16,17-acetal with acetone. Ciclesonide is 11_{β},16_{α},17,21-tetrahydroxypregna-1,4-diene-3,20-dione, cyclic 16,17-acetal with cyclohexanecarboxaldehyde. Mometasone is 9_{α},21-dichloro-11_{β},17-dihydroxy-16_{α}-methylpregna-1,4-diene-3,20-dione. Mometasone furoate is 9_{α},21-dichloro-11_{β},17-dihydroxy-16_{α}-methylpregna-1,4-diene-3,20-dione 17-(2-furoate). RPR 10654 is:

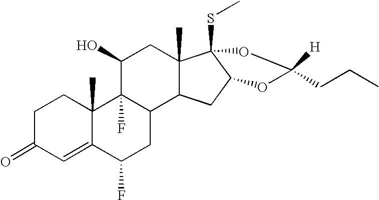

Fluticasone, (6α,11α,16α,17α)-6,9-difluoro-11-hydroxy-16-methyl-3-oxo-17-(1-hydroxy) androsta-1,4-diene-17-carbothioic acid, S-fluoromethyl ester, and fluticasone propionate, (6α,11α,16α,17α-6,9-difluoro-11-hydroxy-16-methyl-3-oxo-17-(1-oxoproxy) androsta-1,4-diene-17-carbothioic acid, S-fluoromethyl ester, are synthetic fluorinated corticosteroids, and are known for topical dermatologic use. Topical corticosteroids constitute a class of primarily synthetic steroids used as anti-inflammatory and antipruritic agents. The mechanism of the anti-inflammatory activity of topical steroids, in general, is unclear. However, corticosteroids are thought to act by the induction of phospholipase A2 inhibitory proteins (lipocortins). It is postulated that these proteins control the biosynthesis of prostaglandins and leukotrienes by inhibiting the release of arachidonic acid.

In one embodiment, the concentrates provided herein contain about 100 to about 10,000 mcg/mL, or about 0.01% to about 1.0% by weight, of one of the above corticosteroids. In another embodiment, the concentrates provided herein contain about 150 to about 4000 mcg/mL of one of the above corticosteroids. In another embodiment, the concentrates provided herein contain about 180 to about 1200 mcg/mL of one of the above corticosteroids. In certain embodiments herein, the corticosteroid is fluticasone propionate, which is present in the concentrates provided herein at the above concentrations.

In another embodiment, the diluted compositions provided herein contain about 10 to about 2000 mcg/mL of one of the above corticosteroids.

In another embodiment, the diluted compositions provided herein contain about 15 to about 1000 mcg/mL of one of the above corticosteroids. In another embodiment, the diluted compositions provided herein contain about 30 to about 300 mcg/mL of one of the above corticosteroids. In certain embodiments herein, the corticosteroid is fluticasone propionate, which is present in the diluted compositions provided herein at the above concentrations.

2. Surfactants

In one embodiment, the surfactants for use in the compositions, including the concentrates and diluted compositions, provided herein are high-HLB (hydrophilic-lipophilic balance) surfactants. Such high-HLB surfactants generally have an HLB greater than about 10. The HLB is a measure on an arbitrary scale of the polarity of a surfactant or mixture of surfactants. In one embodiment, the compositions provided herein contain from about 3% to about 85% by weight of a high-HLB surfactant In one embodiment, the high-HLB surfactant is an ethoxylated derivative of vitamin E such as tocopheryl polyethylene glycol 1000 succinate (TPGS). TPGS has an HLB between about 15 and 19.

In one embodiment, the concentrates provided herein contain about 3% to about 85% by weight of a high-HLB surfactant, such as TPGS. In another embodiment, the concentrates provided herein contain about 5% to about 50% by weight of a high-HLB surfactant, such as TPGS. In another embodiment, the concentrates provided herein contain about 10% to about 40% by weight of a high-HLB surfactant, such as TPGS. In another embodiment, the concentrates provided herein contain about 12% to about 40% by weight of a high-HLB surfactant, such as TPGS.

In another embodiment, the diluted compositions provided herein contain about 0.3% to about 25% by weight of a high-HLB surfactant, such as TPGS. In another embodiment, the diluted compositions provided herein contain about 0.5% to about 12% by weight of a high-HLB surfactant, such as TPGS. In another embodiment, the diluted compositions provided herein contain about 1% to about 10% by weight of a high-HLB surfactant, such as TPGS.

It has been found herein that, at a constant corticosteroid to TPGS ratio, the concentrates provided herein that are more concentrated possess increased stability. As shown in Table 1, below, increasing the concentration of the formulation from 180 mcg/mL of fluticasone in 4% TPGS to 720 mcg/mL fluticasone in 16%TPGS results in an increase in stability of the formulation.

TABLE 1

| | % Fluticasone remaining |
|---|---|
| Formulation | after 1 month at 40° C. |
| 180 mcg/mL Fluticasone in 4% TPGS | 88.2 |
| 540 mcg/mL Fluticasone in 12% TPGS | 95.6 |
| 720 mcg/mL Fluticasone in 16% TPGS | 98.2 |

3. Alkali or Alkaline Earth Metal Halides

The compositions, including the concentrates and diluted compositions provided herein, further contain an alkali or alkaline earth metal salt, including an alkali or alkaline earth metal halide, such as sodium chloride. It has been found herein that the presence of alkali or alkaline earth metal halides, such as sodium chloride, at certain concentrations imparts increased stability to the compositions provided herein, as compared to compositions containing no metal halide. It has also been found herein that the presence of alkali or alkaline earth metal halides, including sodium chloride, at concentrations greater than about 0.9% results in lower stability of the compositions, as compared to compositions containing no metal chloride. See, e.g., Table 2, below.

In one embodiment, the concentrates provided herein contain about 0.01% to about 0.89% by weight of an alkali or alkaline earth metal halide, such as sodium chloride. In another embodiment, the concentrates provided herein contain about 0.1% to about 0.7% by weight of an alkali or alkaline earth metal halide, such as sodium chloride. In another embodiment, the concentrates provided herein contain about 0.4% or 0.5% to about 0.65% by weight of an alkali or alkaline earth metal halide, such as sodium chloride. In another embodiment, the concentrates provided herein contain about 0.6% by weight of an alkali or alkaline earth metal halide, such as sodium chloride.

In another embodiment, the diluted compositions provided herein contain about 0.01% to about 0.6% by weight of an alkali or alkaline earth metal halide, such as sodium chloride. In another embodiment, the diluted compositions provided herein contain about 0.05% to about 0.45% by weight of an alkali or alkaline earth metal halide, such as sodium chloride. In another embodiment, the diluted compositions provided herein contain about 0.05% to about 0.2% or about 0.3% by weight of an alkali or alkaline earth metal halide, such as sodium chloride. In another embodiment, the diluted compositions provided herein contain about 0.1% by weight of an alkali or alkaline earth metal halide, such as sodium chloride.

It has been found herein that the presence of an alkali or alkaline earth metal halide, such as sodium chloride, in the compositions provided herein results in increased stability of the composition. For example, as shown in Table 2 below, the presence of 0.45% NaCl results in an increase in stability relative to control.

TABLE 2

| | Concentration of | % Fluticasone remaining |
|---|---|---|
| Formulation | NaCl (wt %) | after 1 month at 40° C. |
| 180 mcg/ml with 4% | 0% Control^{a} | 92 |
| TPGS | 0.45%^{a} | 95.6 |
| | 0.9%^{a} | 89.8 |
| | 1.8%^{b} | 82.7 |

^{a}pH = 5
^{b}pH = 4.5

4. Other Components

In certain embodiments, the compositions, including the concentrates and diluted compositions, provided herein further contain a buffer, including, but not limited to, citric acid/phosphate, acetate, barbital, borate, Britton-Robinson, cacodylate, citrate, collidine, formate, maleate, Mcllvaine, phosphate, Prideaux-Ward, succinate, citrate-phosphate-borate (Teorell-Stanhagen), veronal acetate, MES (2-(N-morpholino)ethanesulfonic acid), BIS-TRIS (bis(2-hydroxyethyl)iminotris(hydroxymethyl)methane), ADA (N-(2-acetamido)-2-iminodiacetic acid), ACES (N-(carbamoylmethyl)-2-aminoethanesulfonaic acid), PIPES (piperazine-N,N′-bis(2-ethanesulfonic acid)), MOPSO (3-(N-morpholino)-2-hydroxypropanesulfonic acid), BIS-TRIS PROPANE (1,3-bis(tris(hydroxymethyl)methylamino)propane), BES (N,N-bis (2-hydroxyethyl)-2-aminoethanesulfonaic acid), MOPS (3-(N-morpholino) propanesulfonic acid), TES (N-tris(hydroxymethyl)methyl-2-aminoethanesulfonic acid), HEPES (N-(2-hydroxyethyl)piperazine-N′-(2-ethanesulfonic acid), DIPSO (3-(N,N-bis(2-hydroxyethyl)amino)-2-hydroxypropanesulfonic acid), MOBS (4-(N-morpholino)butanesulfonic acid), TAPSO (3-(N-tris(hydroxymethyl)methylamino)-2-hydroxypropanesulfonic acid), TRIZMA® (tris(hydroxymethylaminomethane), HEPPSO (N-(2-hydroxyethyl) piperazine-N′-(2-hydroxypropanesulfonic acid), POPSO (piperazine-N,N′-bis(2-hydroxypropanesulfonic acid)), TEA (triethanolamine), EPPS (N-(2-hydroxyethyl)piperazine-N′-(3-propanesulfonic acid), TRICINE (N-tris(hydroxymethyl)methylglycine), GLY-GLY (glycylglycine), BICINE (N,N-bis (2-hydroxyethyl)glycine), HEPBS (N-(2-hydroxyethyl)piperazine-N′-(4-butanesulfonic acid)), TAPS (N-tris(hydroxymethyl)methyl-3-aminopropanesulfonic acid), AMPD (2-amino-2-methyl-1,3-propanediol), and/or any other buffers known to those of skill in the art. In one embodiment, the buffer is citric acid/phosphate buffer, acetate buffer, citrate buffer or phosphate buffer. In another embodiment, the buffer is a citrate buffer (citric acid/sodium citrate).

The buffer concentration has been found herein to affect the stability of the composition. Buffer concentrations for use in the concentrates provided herein include from about 0.1 mM to about 100 mM, or about 1 mM to about 50 mM, or about 5 mM to about 25 mM. Buffer concentrations for use in the diluted compositions provided herein include from about 0.01 mM to about 25 mM, or about 0.1 mM to about 10 mM, or about 1 mM to about 5 mM.

The pH may also affect the stability of the compositions. In one embodiment, the pH of the composition is about 3 to about 7, or about 4 to about 6, or about 5. In embodiments where the corticosteroid is fluticasone or fluticasone propionate, the compositions are stable in the range of pH from about 4 to about 6.

As shown in Table 3, below, the presence of buffer in the compositions provided herein results in increased stability of the compositions.

TABLE 3

| | | % Fluticasone remaining |
|---|---|---|
| Formulation | Buffer status | after 12 weeks at 40° C. |
| 150 mcg/ml with 4% | Buffered at pH 5.0 | 79.2 |
| TPGS | Unbuffered | 67.4 |

The compositions provided herein also may include excipients and additives. Excipients and additives are any pharmacologically suitable and therapeutically useful substance which is not an active substance. Excipients and additives generally have no pharmacological activity, or at least no undesirable pharmacological activity. The excipients and additives include, but are not limited to, stabilizers, complexing agents, antioxidants, or preservatives which prolong the duration of use of the finished pharmaceutical formulation, flavorings, vitamins, or other additives known in the art.

Complexing agents include, but are not limited to, ethylenediaminetetraacetic acid (EDTA) or a salt thereof, such as the disodium salt, citric acid, nitrilotriacetic acid and the salts thereof. In one embodiment, the complexing agent is EDTA. In another embodiment, the complexing agent is sodium edetate. In these embodiments, the compositions contain sodium edetate at a concentration of about 0.05 mg/mL to about 0.5 mg/mL, or about 0.1 mg/mL to about 0.2 mg/mL.

Preservatives include, but are not limited to, those that protect the solution from contamination with pathogenic particles, including benzalkonium chloride or benzoic acid, or benzoates such as sodium benzoate. Antioxidants include, but are not limited to, vitamins, provitamins, ascorbic acid, vitamin E or salts or esters thereof.

Stabilizers include ferric citrate, ferric phosphate, ferric pyrophosphate and other ferric salts known in the art. As shown in Table 4, below, the presence of ferric citrate increases the stability of the compositions provided herein.

TABLE 4

| | Concentration of | % Fluticasone remaining |
|---|---|---|
| Formulation | ferric citrate | after 4 weeks at 40° C. |
| 180 mcg/ml with | 0 (Control) | 92.3 |
| 4% TPGS | 2 mcg/mL | 93.0 |
| | 10 mcg/mL | 95.6 |

The compositions provided herein also may include a cosolvent, which increases the solubility of additives or the active ingredient(s). Cosolvents for use herein include, but are not limited to, hydroxylated solvents or other polar solvents, such as alcohols such as isopropyl alcohol, glycols such as propylene glycol, polyethylene glycol, polypropylene glycol, glycol ether, glycerol, and polyoxyethylene alcohols. In certain embodiments herein, the compositions contain a glycol. In other embodiments, the compositions contain propylene glycol and/or polyethylene glycol, including polyethylene glycol 300 or 400.

In certain embodiments, the concentrates provided herein contain a glycol at a concentration of about 0% to about 50%, or about 1% to about 40%, or about 6% to about 20%, by weight. In one embodiment, the glycol is propylene glycol. In another embodiment, the glycol is polyethylene glycol 300 or 400.

In other embodiments, the diluted compositions provided herein contain a glycol at a concentration of about 0% to about 15%, or about 0.1% to about 10%, or about 1% to about 4 or 5%, by weight. In one embodiment, the glycol is propylene glycol. In another embodiment, the glycol is polyethylene glycol 300 or 400.

The compositions provided herein may also contain one or more emulsifiers. Emulsifiers for use herein include, but are not limited to, polyoxyetheylene sorbitan fatty esters or polysorbates, including, but not limited to, polyethylene sorbitan monooleate (Polysorbate 80), polysorbate 20 (polyoxyethylene (20) sorbitan monolaurate), polysorbate 65 (polyoxyethylene (20) sorbitan tristearate), polyoxyethylene (20) sorbitan mono-oleate, polyoxyethylene (20) sorbitan monopalmitate, polyoxyethylene (20) sorbitan monostearate; lecithins; alginic acid; sodium alginate; potassium alginate; ammonium alginate; calcium alginate; propane-1,2-diol alginate; agar; carrageenan; locust bean gum; guar gum; tragacanth; acacia; xanthan gum; karaya gum; pectin; amidated pectin; ammonium phosphatides; microcrystalline cellulose; methylcellulose; hydroxypropylcellulose; hydroxypropylmethylcellulose; ethylmethylcellulose; carboxymethylcellulose; sodium, potassium and calcium salts of fatty acids; mono- and di-glycerides of fatty acids; acetic acid esters of mono- and di-glycerides of fatty acids; lactic acid esters of mono- and di-glycerides of fatty acids; citric acid esters of mono- and di-glycerides of fatty acids; tartaric acid esters of mono- and di-glycerides of fatty acids; mono- and diacetyltartaric acid esters of mono- and di-glycerides of fatty acids; mixed acetic and tartaric acid esters of mono- and di-glycerides of fatty acids; sucrose esters of fatty acids; sucroglycerides; polyglycerol esters of fatty acids; polyglycerol esters of polycondensed fatty acids of castor oil; propane-1,2-diol esters of fatty acids; sodium stearoyl-2-lactylate; calcium stearoyl-2-lactylate; stearoyl tartrate; sorbitan monostearate; sorbitan tristearate; sorbitan monolaurate; sorbitan mono-oleate; sorbitan monopalmitate; extract of quillaia; polyglycerol esters of dimerised fatty acids of soya bean oil; oxidatively polymerised soya bean oil; and pectin extract.

In certain embodiments herein, the emulsifier(s) is (are) a polyoxyetheylene sorbitan fatty ester or polysorbate, including, but not limited to, polyethylene sorbitan monooleate (Polysorbate 80), polysorbate 20 (polyoxyethylene (20) sorbitan monolaurate), polysorbate 65 (polyoxyethylene (20) sorbitan tristearate), polyoxyethylene (20) sorbitan mono-oleate, polyoxyethylene (20) sorbitan monopalmitate, polyoxyethylene (20) sorbitan monostearate; sorbitan monostearate; sorbitan tristearate; sorbitan monolaurate; sorbitan mono-oleate; or sorbitan monopalmitate. In further embodiments, the emulsifier(s) is (are) polysorbate 80, sorbitan monolaruate or polyoxyethylene (20) sorbitan monolaurate.

In another embodiment, the concentrates provided herein contain about 0% to about 50%, or about 1% to about 40%, or about 6% to about 20%, by weight of glycerin. In another embodiment, the diluted compositions provided herein contain about 0% to about 15%, or about 1% to about 10%, or about 1% to about 5%, by weight of glycerin.

C. Preparation of Compounds for Use in the Compositions

The preparation of the compounds used in the compositions provided herein is described below. Any such compound or similar compound may be synthesized according to a method discussed in general below or by only minor modification of the methods by selecting appropriate starting materials.

Fluticasone propionate may be synthesized by the procedure disclosed in U.S. Pat. No. 4,335,121. Briefly, the corresponding carbothioic acid is prepared from the carboxylic acid precursor by reaction with dimethylthiocarbamoyl chloride in the presence of triethylamine. Reaction with bromochloromethane and sodium hydrogen carbonate in dimethylacetamide affords the corresponding S-chloromethyl carbothioate. This compound is treated with sodium iodide in acetone to give the corresponding S-iodomethyl carbothioate. Fluoride substitution of the iodo group is accomplished by reaction with silver fluoride in acetonitrile to afford the desired compound.

Beclomethasone may be synthesized by the procedure in U.S. Pat. No. 4,041,055. Briefly, reaction of a steroidal 17_{β}-hydroxy-17_{α}-ethynyl compound with phenylsulfenyl chloride to form an allenyl sulfoxide, addition of methoxide to the allenyl sulfoxide, reacting the resulting product with trimethylphosphite, and adding a peracid to the resulting compound.

Flunisolide and triamcinolone acetonide may be synthesized by the procedure in U.S. Pat. No. 3,126,375. Briefly, reaction of 6_{α}-fluoro-11_{β},16_{α},17,21-tetrahydroxy-pregna-1,4-diene-3,20-dione or 9-fluoro-11_{β},16_{α},17,21-tetrahydroxypregna-1,4-diene-3,20-dione (U.S. Pat. No. 2,997,489) with acetone and copper sulfate provides the 16_{α},17-cyclic acetonides. Similarly, ciclesonide may be prepared by reacting 11_{β},16_{α}-17,21-tetrahydroxypregna-1,4-diene-3,20-with cyclohexanecarboxaldehyde in the presence of copper sulfate.

Mometasone my be synthesized by the procedure in U.S. Pat. Nos. 5,750,745, 5,616,742 and 5,502,222. Briefly, reaction of 9_{α},21-dihydroxy-11_{β},17-dihydroxy-16α-methylpregna-1,4-diene-3,20-dione with triphenylphosphine and carbon tetrachloride, p-toluenesulfonyl chloride and LiCl, or methanesulfonyl chloride and LiCl produces the desired dichloro compound.

D. Formulation of Pharmaceutical Compositions

The compositions provided herein are prepared by procedures described below and in the Examples. It has been found herein that certain process parameters influence the stability of the resulting composition, including the concentrates and diluted compositions. Such process parameters include order of addition of cosolvent, such as a glycol; temperature of the concentrate; temperature of the aqueous phase; and filtration and filter size. In addition, order of mixing of the concentrate and the aqueous phase influences the concentration of drug that may be achieved in the diluted compositions.

1. Order of Addition of Drug and Mixing of Concentrate and Aqueous Phase

In one embodiment, the drug is dissolved in the surfactant, such as molten TPGS, and then added to water. For example, when fluticasone propionate is prepared this way with a target concentration of 180 mcg/ml, the amount solubilized by addition of molten TPGS containing the drug to water is about 180 mcg/mL. The amount solubilized by addition of water to molten TPGS, followed by addition of the drug, is only 77 mcg/mL. If the aqueous phase is added to the molten TPGS, the TPGS phase forms a gel and this, apart from making the mixing process slower, makes the amount solubilized lower and variable.

As shown in Table 5, below, if the drug is first dissolved in molten TPGS, the order of mixing with aqueous phase also affects the solubility of the drug. Thus, in Process 1, aqueous phase is added to a solution of the drug in molten TPGS. In Process 2, a solution of the drug in molten TPGS is added to the aqueous phase. To avoid gel formation, the TPGS phase is added to the aqueous phase and slowly. Rapid addition may form gel. Also, high speed mixing reduces the formation of gel.

TABLE 5

| | | Amount solubilized as | |
|---|---|---|---|
| Process for Preparation | | percent of theoretical | |
| of Fluticasone | Target | concentration | |
| Compositions | concentration | Mean | % RSD |
| Process 1 (Aqueous | 180 mcg/ml | 93.64 | 5.42 |
| phase added to the | | | |
| TPGS phase all at once | | | |
| and then mixed) | | | |
| Process 2 (TPGS phase | 180 mcg/ml | 99.53 | 1.40 |
| added to aqueous phase | | | |
| slowly with mixing) | | | |

2. Order of Addition of Cosolvent

Whether the cosolvent, if present, is added to either the molten TPGS phase or the aqueous phase prior to mixing of the concentrate and the aqueous phase has been found herein to affect the stability of the resulting diluted composition. As shown in Table 6a, below, adding the cosolvent to the molten TPGS phase results in a more stable diluted composition. Cosolvents such as propylene glycol (PG) and polyethylene glycol 400 (PEG 400) can influence the amount solubilized by affecting the amount dissolved in the molten TPGS phase.

TABLE 6a

| | % Fluticasone remaining |
|---|---|
| Addition to 180 mcg/ml formulation | after 1 month at 40° C. |
| Propylene glycol added to molten | 93.87 |
| TPGS phase | |
| Propylene glycol added to buffered | 90.42 |
| aqueous phase | |

However, the order of addition influences the amount of drug that may be solubilized. If PG or PEG 400 is added in the molten TPGS phase before adding the drug, the amount solubilized increases (see Table 6b, below).

TABLE 6b

| | Fluticasone solubilized in |
|---|---|
| | molten surfactant phase at 60° C. |
| Surfactant phase | (mg/g of TPGS) |
| TPGS alone | 6.5 |
| TPGS and propylene glycol (2.4:1) | 11.9 |
| TPGS and polyethylene glycol 400 | 20.0 |
| (1:1) | |

If PG or PEG 400 is added to the aqueous phase before or after mixing with the molten TPGS phase, then amount solubilized is not significantly affected.

3. Temperature of the Concentrate

It has also been found herein that the temperature of the molten TPGS phase during mixing with aqueous phase affects the stability of the diluted composition. As shown in Table 7, below, maintaining the molten TPGS phase at 60° C. instead of 45° C. results in a more stable diluted composition.

TABLE 7

| | Temperature of | |
|---|---|---|
| Fluticasone | molten TPGS | % remaining after |
| Formulation | phase | storage at 50° C. |
| 110 mcg/ml; TPGS 2%, | 45° C. | 4 weeks: 60.7% |
| Polyethylene glycol 400 | 60° C. | 4 weeks: 66.9% |
| 2% | | |
| 220 mcg/ml: TPGS 4% | 45° C. | 1 week: 80.0 |
| | 60° C. | 1 week: 83.8 |

4. Temperature of the Aqueous Phase

The temperature of the aqueous phase during mixing with the molten TPGS phase has also been found herein to affect the stability of the diluted compostion. For example, as shown in Table 8, maintaining the aqueous phase at ambient temperature results in an increase in stability relative to maintaining the aqueous phase at 50° C. In preparations where the temperature of the aqueous phase was equal to the temperature of the molten phase, the solubilized drug precipitated quicker and in larger amounts.

Thus, maintaining the aqueous phase at a temperature lower than the molten TPGS phase temperature, e.g., ambient temperature or lower, such as 1° C. to 25° C., or at 5° C., or at 20° C., results in a more stable diluted composition.

TABLE 8

| | Temperature of the | % Fluticasone remaining |
|---|---|---|
| Formulation | aqueous phase | after 8 weeks at 40° C. |
| 180 mcg/ml | Ambient temperature | 86.7 |
| with 4% TPGS | (˜22° C.) | |
| | 50° C. | 74.0 |

5. Filtration and Filter Size

Filtration of the diluted composition also has been found herein to affect the stability of the diluted composition. As shown in Tables 9 and 10, below, filtration with filter pore size as low as about 0.1 μm, or about 0.22 μm, results in increased stability of the diluted composition.

TABLE 9

| | | % Fluticasone remaining |
|---|---|---|
| Formulation | Filtration status | after 3 weeks at 40° C. |
| 180 mcg/ml with 4% | Filtered (0.22 μm) | 86.5 |
| TPGS | Unfiltered | 66.1 |

TABLE 10

| | | % Fluticasone remaining |
|---|---|---|
| Formulation | Filter size | after 1 month at 40° C. |
| 180 mcg/ml with | 0.1 μm | 101.04 |
| 4% TPGS | 0.22 μm | 85.79 |
| | 0.8 μm | 69.20 |

E. Combination and Kits

Combinations and kits containing the compositions, packaged into suitable packaging material are provided. A kit typically includes a label or packaging insert including a description of the components or instructions for use (e.g., for treatment of a bronchconstrictive disorder) of the components therein. A kit can contain a collection of such components.

Kits therefore optionally include labels or instructions for using the kit components in a method provided herein. Instructions can include instructions for practicing any of the methods.

The instructions can be on “printed matter,” e.g., on paper or cardboard within the kit, or on a label affixed to the kit or packaging material, or attached to a vial or tube containing a component of the kit. Instructions can additionally be included on a computer readable medium, such as a disk (floppy diskette or hard disk), optical CD such as CD- or DVD-ROM/RAM, magnetic tape, electrical storage media such as RAM and ROM and hybrids of these such as magnetic/optical storage media. Kits can additionally include buffering agent, a preservative, or a stabilizing agent. Each component of the kit can be enclosed within an individual container and all of the various containers can be within a single package. Kits can be designed for cold storage.

F. Evaluation of the Activity of the Compositions

Standard physiological, pharmacological and biochemical procedures are available for testing the compositions provided herein to identify those that possess bronchdilatory activity.

In vitro and in vivo assays that may be used to evaluate bronchodilatory activity are well known to those of skill in the art. See also, e.g., U.S. Pat. Nos. 3,994,974, and 6,068,833; German Patent No. 2,305,092; Kaumann et al. (1985) Naunyn-Schmied Arch. Pharmacol. 331:27-39; Lemoine et al. (1985) Naunyn-Schmied Arch. Pharmacol. 331:40-51; Tomioka et al. (1981) Arch. Int. Pharmacodyn. 250:279-292; Dellamary et al. (2000) Pharm. Res. 17(2):168-174; Rico-Mendez et al. (1999) Rev. Alerg. Mex. 46(5):130-135; Seberova et al. (2000) Respir. Med. 94(6):607-611; Lotvall et al (1999) Can. Respir. J. 6(5):412-416; Campbell et al. (1999) Respir. Med. 93(4)236-244; Nightingale et al. (1999) Am. J. Respir. Crit. Care Med. 159(6):1786-1790; Lecaillon et al. (1999) Eur. J. Clin. Pharmacol. 55(2):131-138; Bartow et al. (1998) Drugs 55(2):303-322; Ekstrom et al. (1998) Respir. Med. 92(8):1040-1045; Ringdal et al. (1998) Respir. Med. 92(8):1017-1021; Totterman et al. (1998) Eur. Respir. J. 12(3):573-579; Palmqvist et al (1997) Eur. Respir. J. 10(11):2484-2489; Nielsen et al. (1997) Eur. Respir J. 10(9):2105-2109; Ullman et al. (1996) Allergy 51(10):745-748; Selroos et al. (1996) Clin. Immunother. 6:273-299; and Schreurs et al. (1996) Eur. Respir. J. 9(8):1678-1683.

G. Methods of Treatment of Bronchoconstrictive Disorders

The diluted compositions provided herein are used for treating, preventing, or ameliorating one or more symptoms of a bronchoconstrictive disorders in a mammal. In one embodiment, the method includes administering to a mammal an effective amount of a composition containing (i) a corticosteroid anti-inflammatory agent such as beclomethasone dipropionate (BDP), beclomethasone monopropionate (BMP), flunisolide, triamcinolone acetonide, ciclesonide, mometasone, mometasone furoate (Asmanex®) Twisthaler™, Schering-Plough Corporation, Kenilworth, N.J.), RPR 106541, fluticasone or fluticasone propionate; (ii) a surfactant; (iii) sodium chloride; and (iv) water, whereby the disease or disorder is treated or prevented, or one or more symptoms are ameliorated. The mammal treated is, in certain embodiments, a human.

In another embodiment, the method provided herein includes oral or nasal administration of a composition provided herein. In certain embodiments herein, the composition is directly administered to a patient in need of such treatment via nebulization without dilution or other modification of the composition prior to administration.

The methods for treatment, prevention, or amelioration of one or more symptoms of bronchoconstrictive disorders, in another embodiment, further include administering one or more of (a) or (b) as follows: (a) a β₂-adrenoreceptor agonist; or (b) a dopamine (D₂) receptor agonist; simultaneously with, prior to or subsequent to the composition provided herein.

β₂-Adrenoreceptor agonists for use in combination with the compositions provided herein include, but are not limited to, Albuterol (α¹-(((1,1-dimethylethyl)amino)methyl)-4-hydroxy-1,3-benzenedimethanol); Bambuterol (dimethylcarbamic acid 5-(2-((1,1-dimethylethyl)amino)-1-hydroxyethyl)-1,3-phenylene ester); Bitolterol (4-methylbenzoic acid 4-(2-((1,1-dimethylethyl)amino)-1-hydroxyethyl)-1,2-phenylene ester); Broxaterol (3-bromo-α-(((1,1-dimethylethyl)amino)methyl)-5-isoxazolemethanol); Isoproterenol (4-(1-hydroxy-2-((1-methylethyl)amino)ethyl)-1,2-benzenediol); Trimetoquinol (1,2,3,4-tetrahydro-1-((3,4,5-trimethoxyphenyl)methyl)-6,7-isoquinolinediol); Clenbuterol (4-amino-3,5-dichloro-α-(((1,1-diemthylethyl) amino)methyl)benzenemethanol); Fenoterol (5-(1-hydroxy-2-((2-(4-hydroxyphenyl)-1-methylethyl)amino)ethyl)-1,3-benzenediol); Formoterol (2-hydroxy-5-((1RS)-1-hydroxy-2-(((1RS)-2-(p-methoxyphenyl)-1-methylethyl)amino)ethyl)formanilide); (R,R)-Formoterol; Desformoterol ((R,R) or (S,S)-3-amino-4-hydroxy-α-(((2-(4-methoxyphenyl)-1-methylethyl)amino)methyl)benzenemethanol); Hexoprenaline (4,4′-(1,6-hexanediyl)-bis(imino(1-hydroxy-2,1-ethanediyl)))bis-1,2-benzenediol); Isoetharine (4-(1-hydroxy-2-((1-methylethyl)amino)butyl)-1,2-benzenediol); Isoprenaline (4-(1-hydroxy-2-((1-methylethyl)amino)ethyl)-1,2-benzenediol); Metaproterenol (5-(1-hydroxy-2-((1-methylethyl)amino)ethyl)-1,3-benzenediol); Picumeterol (4-amino-3,5-dichloro-α-(((6-(2-(2-pyridinyl)ethoxy)hexyl)amino)methyl)benzenemethanol); Pirbuterol ((α⁶-(((1,1-dimethylethyl)amino)methyl)-3-hydroxy-2,6-pyridinemethanol); Procaterol (((R*,S*)-(±)-8-hydroxy-5-(1-hydroxy-2-((1-methylethyl)amino)butyl)-2(1H)-quinolinone); Reproterol ((7-(3-((2-(3,5-dihydroxyphenyl)-2-hydroxyethyl)amino)propyl)-3,7-dihydro-1,3-dimethyl-1H-purine-2,6-dione); Rimiterol (4-(hydroxy-2-piperidinylmethyl)-1,2-benzenediol); Salbutamol ((±)-á¹-(((1,1-dimethylethyl)amino)methyl)-4-hydroxy-1,3-benzenedimethanol); (R)-Salbutamol; Salmeterol ((±)-4-hydroxy-α¹-(((6-(4-phenylbutoxy)hexyl)amino)methyl)-1,3-benzenedimethanol); (R)-Salmeterol; Terbutaline (5-(2-((1,1-dimethylethyl)amino)-1-hydroxyethyl)-1,3-benzenediol); Tulobuterol (2-chloro-α-(((1,1-dimethylethyl)amino)methyl)benzenemethanol); and TA-2005 (8-hydroxy-5-((1R)-1-hydroxy-2-(N-((1R)-2-(4-methoxyphenyl)-1-methylethyl)amino)ethyl)carbostyril hydrochloride).

Dopamine (D₂) receptor agonists include, but are not limited to, Apomorphine ((R)-5,6,6a,7-tetrahydro-6-methyl-4H-dibenzo[de,g]quinoline-10,11-diol); Bromocriptine ((5′α)-2-bromo-12′-hydroxy-2′-(1-methylethyl)-5′-(2-methylpropyl)ergotaman-3′,6′,18-trione); Cabergoline ((8â)-N-(3-(dimethylamino)propyl)-N-((ethylamino)carbonyl)-6-(2-propenyl)ergoline-8-carboxamide); Lisuride (N′-((8α)-9,10-didehydro-6-methylergolin-8-yl)-N,N-diethylurea); Pergolide ((8α)-8-((methylthio)methyl)-6-propylergoline); Levodopa (3-hydroxy-L-tryrosine); Pramipexole((S)-4,5,6,7-tetrahydro-N⁶-propyl-2,6-benzothiazolediamine); Quinpirole hydrochloride(trans-(−)-4aR-4,4a,5,6,7,8,8a,9-octahydro-5-propyl-1H-pyrazolo[3,4-g]quinoline hydrochloride); Ropinirole (4-(2-(dipropylamino)ethyl)-1,3-dihydro-2H-indol-2-one); and Talipexole (5,6,7,8-tetrahydro-6-(2-propenyl)-4H-thiazolo[4,5-d]azepin-2-amine). Other dopamine D₂ receptor agonists for use herein are disclosed in International Patent Application Publication No. WO 99/36095.

Other active ingredients for use herein in combination therapy, include, but are not limited to, IL-5 inhibitors such as those disclosed in U.S. Pat. Nos. 5,668,110, 5,683,983, 5,677,280 and 5,654,276; antisense modulators of IL-5 such as those disclosed in U.S. Pat. No. 6,136,603; milrinone (1,6-dihydro-2-methyl-6-oxo-[3,4′-bipyridine]-5-carbonitrile); milrinone lactate; tryptase inhibitors such as those disclosed in U.S. Pat. No. 5,525,623; tachykinin receptor antagonists such as those disclosed in U.S. Pat. Nos. 5,691,336, 5,877,191, 5,929,094, 5,750,549 and 5,780,467; leukotriene receptor antagonists such as montelukast sodium (Singular®, R-(E)]-1-[[[1-[3-[2-(7-chloro-2-quinolinyl)ethenyl]phenyl]-3-[2-(-1-hydroxy-1-methylethyl)phenyl]propyl]thio]methyl]cyclopropaneacetic acid, monosodium salt), 5-lypoxygenase inhibitors such as zileuton (Zyflo®, Abbott Laboratories, Abbott Park, Ill.), and anti-IgE antibodies such as Xolair® (recombinant humanized anti-IgE monoclonal antibody (CGP 51901; IGE 025A; rhuMAb-E25), Genentech, Inc.).

The bronchoconstrictive disorder to be treated, prevented, or whose one or more symptoms are to be ameliorated is associated with asthma, including, but not limited to, bronchial asthma, allergic asthma and intrinsic asthma, e.g., late asthma and airway hyper-responsiveness; chronic bronchitis; and other chronic obstructive pulmonary diseases.

H. Nebulizers

The compositions provided herein are intended for administration to a patient in need of such treatment via nebulization. Nebulizers that nebulize liquid formulations containing no propellant are suitable for use with the compositions provided herein. Nebulizers are available from, e.g, Pari GmbH (Starnberg, Germany), DeVilbiss Healthcare (Heston, Middlesex, UK), Healthdyne, Vital Signs, Baxter, Allied Health Care, Invacare, Hudson, Omron, Bremed, AirSep, Luminscope, Medisana, Mountain Medical, Aerosol Medical Ltd. (Colchester, Essex, UK), AFP Medical (Rugby, Warwickshire, UK), Bard Ltd. (Sunderland, UK), Carri-Med Ltd. (Dorking, UK), Plaem Nuiva (Brescia, Italy), Henleys Medical Supplies (London, UK), Intersurgical (Berkshire, UK), Lifecare Hospital Supplies (Leies, UK), Medic-Aid Ltd. (West Sussex, UK), Medix Ltd. (Essex, UK), Sinclair Medical Ltd. (Surrey, UK), and many others.

Nebulizers for use herein include, but are not limited to, jet nebulizers (optionally sold with compressors), ultrosonic nebulizers, and others. Exemplary jet nebulizers for use herein include Pan LC plus/ProNeb, Pari LC plus/ProNeb Turbo, Pari LC plus/Dura Neb 1000 & 2000, Pari LC plus/Walkhaler, Pari LC plus/Pari Master, Pari LC star, Plumo-aide, Pulmo-aide LT, Pulmo-aide traveler, Invacare Passport, Inspiration Healthdyne 626, Pulmo-Neb Traverler, DeVilbiss 646, Whisper Jet, Acorn II, Misty-Neb, Allied aerosol, Schuco Home Care, Lexan Plasic Pocet Neb, SideStream Hand Held Neb, Mobil Mist, Up-Draft, Up-Draft II, T Up-Draft, ISO-NEB, AVA-NEB, Micro Mist, and PulmoMate. Exemplary ultrasonic nebulizers for use herein include MicroAir, UltraAir, CompAir, Pulmosonic, Scout, 5003 Ultrasonic Neb, 5110 Ultrasonic Neb, 5004 Desk Ultrasonic Nebulizer, Mystique Ultrasonic, Luminscope's Ultrasonic Nebulizer, Medisana Ultrasonic Nebulizer, Microstat Ultrasonic Nebulizer, and MABISMist Hand Held Ultrasonic Nebulizer. Other nebulizers for use herein include 5000 Electromagnetic Neb, 5001 Electromagnetic Neb 5002 Rotary Piston Neb, Lumineb I Piston Nebulizer 5500, and AeroEclipse Breath Actuated Nebulizer.

1. Articles of Manufacture

The compositions provided herein may be packaged as articles of manufacture containing packaging material, a composition provided herein, which is useful for treatment, prevention or amelioration of one or more symptoms of diseases or disorders associated with undesired and/or uncontrolled bronchoconstriction, and a label that indicates that the composition is used for treatment, prevention or amelioration of one or more symptoms of diseases or disorders associated with undesired and/or uncontrolled bronchoconstriction.

The articles of manufacture provided herein contain packaging materials. Packaging materials for use in packaging pharmaceutical products are well known to those of skill in the art. See, e.g., U.S. Pat. Nos. 5,323,907, 5,052,558 and 5,033,252. Examples of pharmaceutical packaging materials include, but are not limited to, blister packs, bottles, tubes, inhalers, pumps, bags, vials, containers, syringes, bottles, and any packaging material suitable for a selected formulation and intended mode of administration and treatment.

In one embodiment herein, the compositions are packaged with a nebulizer for direct administration of the composition to a patient in need thereof. In another embodiment, the compositions are concentrates that are packaged with a nebulizer, and are diluted prior to administration to a patient in need thereof.

The following examples are included for illustrative purposes only and are not intended to limit the scope of the invention.

### EXAMPLE 1

The following are exemplary compositions provided herein. The concentrate is intended to be diluted to form the diluted composition prior to administration to a patient in need thereof.

| Ingredient | Concentrate | Diluted composition |
|---|---|---|
| Fluticasone | 900 mcg/ml | 150 mcg/ml |
| propionate | | |
| TPGS | 24% w/w | 4% w/w |
| Propylene glycol | 10% w/w | 1.67% w/w |
| Glycerin | 12% w/w | 2% w/w |
| NaCl | 0.6% w/w | 0.1% w/w |
| Buffer | 12 mM | 2 mM |
| Purified water | 52.7% w/w | 92.1% w/w |

Since modifications will be apparent to those of skill in this art, it is intended that this invention be limited only by the scope of the appended claims.

What is claimed is:

1. A composition, comprising:
(i) a corticosteroid anti-inflammatory agent selected from beclomethasone dipropionate (BDP), beclomethasone monopropionate (BMP), flunisolide, triamcinolone acetonide, ciclesonide, mometasone, mometasone furoate, RPR 106541 having the formula: 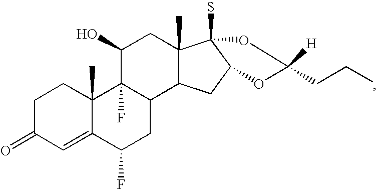
fluticasone or fluticasone propionate;
(ii) a surfactant;
(iii) an alkali or alkaline earth metal salt at a concentration of about 0.01% to about 0.89% by weight; and
(iv) water.

2. The composition of claim 1, wherein the corticosteroid anti-inflammatory agent is fluticasone or fluticasone propionate.

3. The composition of claim 1, comprising about 1% to about 95% by weight water.

4. The composition of claim 1, comprising about 15% to about 85% by weight water.

5. The composition of claim 1, comprising about 30% to about 80% water.

6. The composition of claim 1, comprising about 5% to about 99% water.

7. The composition of claim 1, comprising about 60% to about 99% water.

8. The composition of claim 1, comprising about 80% to about 99% water.

9. The composition of claim 1, comprising about 40% to about 90% water.

10. The composition of claim 1, comprising about 100 to about 10,000 mcg/mL of the corticosteroid.

11. The composition of claim 1, comprising about 150 to about 4000 mcg/mL of the corticosteroid.

12. The composition of claim 1, comprising about 180 to about 1200 mcg/mL of the corticosteroid.

13. The composition of claim 1, comprising about 10 to about 2000 mcg/mL of the corticosteroid.

14. The composition of claim 1, comprising about 15 to about 1000 mcg/mL of the corticosteroid.

15. The composition of claim 1, comprising about 30 to about 300 mcg/mL of the corticosteroid.

16. The composition of claim 1, wherein the surfactant is a high-hydrophilic-lipophilic balance (HLB) surfactant.

17. The composition of claim 16, wherein the HLB of the surfactant is greater than about 10.

18. The composition of claim 1, comprising about 3% to about 85% by weight of a high-HLB surfactant.

19. The composition of claim 18, wherein the high-HLB surfactant is an ethoxylated derivative of vitamin E.

20. The composition of claim 19, wherein the high-HLB surfactant is tocopheryl polyethylene glycol 1000 succinate (TPGS).

21. The composition of claim 1, comprising about 5% to about 50% by weight of a high-HLB surfactant.

22. The composition of claim 1, comprising about 10% to about 40% by weight of a high-HLB surfactant.

23. The composition of claim 1, comprising about 12% to about 40% by weight of a high-HLB surfactant.

24. The composition of claim 1, comprising about 0.3% to about 25% by weight of a high-HLB surfactant.

25. The composition of claim 1, comprising about 0.5% to about 12% by weight of a high-HLB surfactant.

26. The composition of claim 1, comprising about 1% to about 10% by weight of a high-HLB surfactant.

27. The composition of claim 1, wherein the alkaline earth metal salt is a halide selected from magnesium and calcium halides.

28. The composition of claim 1, wherein the alkaline earth metal salt is magnesium chloride or calcium chloride.

29. The composition of claim 1 that comprises an alkali metal salt.

30. The composition of claim 29, wherein the alkali metal salt is a halide selected from sodium, lithium, and potassium halides.

31. The composition of claim 1, wherein the alkali metal salt is sodium chloride, lithium chloride or potassium chloride.

32. The composition of claim 1, wherein the alkali metal salt is sodium chloride.

33. The composition of claim 1, comprising about 0.01% to about 0.89% by weight of the alkali or alkaline earth metal salt.

34. The composition of claim 1, comprising about 0.1% to about 0.7% by weight of the alkali or alkaline earth metal salt.

35. The composition of claim 1, comprising about 0.4% or 0.5% to about 0.65% by weight of the alkali or alkaline earth metal salt.

36. The composition of claim 1, comprising about 0.6% by weight of an alkali or alkaline earth metal salt.

37. The composition of claim 1, comprising about 0.01% to about 0.6% by weight of the alkali or alkaline earth metal salt.

38. The composition of claim 1, comprising about 0.05% to about 0.45% by weight of an alkali or alkaline earth metal salt.

39. The composition of claim 1, comprising about 0.05% to about 0.3% by weight of the alkali or alkaline earth metal salt.

40. The composition of claim 1, comprising about 0.05% to about 0.2% by weight of the alkali or alkaline earth metal salt.

41. The composition of claim 1, comprising about 0.1% by weight of the alkali or alkaline earth metal salt.

42. The composition of claim 1, comprising about 0.45% by weight of the alkali or alkaline earth metal salt.

43. The composition of claim 1, further comprising a buffer.

44. The composition of claim 43, wherein the buffer is citric acid/phosphate, acetate, barbital, borate, Britton-Robinson, cacodylate, citrate, collidine, formate, maleate, Mcllvaine, phosphate, Prideaux-Ward, succinate, citrate-phosphate-borate (Teorell-Stanhagen), veronal acetate, MES (2-(N-morpholino)ethanesulfonic acid), BIS-TRIS (bis(2-hydroxyethyl)iminotris(hydroxymethyl)methane), ADA (N-(2-acetamido)-2-iminodiacetic acid),ACES (N-(carbamoylmethyl)-2-aminoethanesulfonaic acid), PIPES (piperazine-N,N′-bis(2-ethanesulfonic acid)), MOPSO (3-(N-morpholino)-2-hydroxypropanesulfonic acid),BIS-TRIS PROPANE (1,3-bis(tris(hydroxymethyl)methylamino)propane), BES (N,N-bis(2-hydroxyethyl)-2-aminoethanesulfonaic acid), MOPS (3-(N-morpholino)propanesulfonic acid), TES (N-tris(hydroxymethyl)methyl-2-aminoethanesulfonic acid), HEPES (N-(2-hydroxyethyl)piperazine-N′-(2-ethanesulfonic acid), DIPSO (3-(N,N-bis(2-hydroxyethyl)amino)-2-hydroxypropanesulfonic acid), MOBS (4-(N-morpholino)butanesulfonic acid), TAPSO (3-(N-tris(hydroxymethyl)methylamino)-2-hydroxypropanesulfonic acid), TRIZMA® (tris(hydroxymethylaminomethane), HEPPSO (N-(2-hydroxyethyl)piperazine-N′-(2-hydroxypropanesulfonic acid), POPSO (piperazine-N,N′-bis(2-hydroxypropanesulfonic acid)), TEA (triethanolamine), EPPS (N-(2-hydroxyethyl)piperazine-N′-(3-propanesulfonic acid), TRICINE (N-tris(hydroxymethyl)methylglycine), GLY-GLY (glycylglycine), BICINE (N,N-bis(2-hydroxyethyl)glycine), HEPBS (N-(2-hydroxyethyl)piperazine-N′-(4-butanesulfonic acid)), TAPS (N-tris(hydroxymethyl)methyl-3-aminopropanesulfonic acid), or AMPD (2-amino-2-methyl-1,3-propanediol).

45. The composition of claim 43, wherein the buffer is citric acid/phosphate buffer, acetate buffer, citrate buffer or phosphate buffer.

46. The composition of claim 43, wherein the buffer is a citrate buffer (citric acid/sodium citrate).

47. The composition of claim 43, wherein the buffer concentration is about 0.1 mM to about 100 mM.

48. The composition of claim 43, wherein the buffer concentration is about 1 mM to about 50 mM.

49. The composition of claim 43, wherein the buffer concentration is about 5 mM to about 25 mM.

50. The composition of claim 43, wherein the buffer concentration is about 0.01 mM to about 25 mM.

51. The composition of claim 43, wherein the buffer concentration is about 0.1 mM to about 10 mM.

52. The composition of claim 43, wherein the buffer concentration is about 1 mM to about 5 mM.

53. The composition of claim 1, further comprising an excipient or additive.

54. The composition of claim 53, wherein the excipient or additive is a stabilizer, complexing agent, antioxidant, preservative, flavoring or vitamin.

55. The composition of claim 54, wherein the complexing agent is ethylenediaminetetraacetic acid (EDTA) or a salt thereof.

56. The composition of claim 54, wherein the complexing agent is EDTA.

57. The composition of claim 54, wherein the preservative is benzalkonium chloride, benzoic acid, or sodium benzoate.

58. The composition of claim 54, wherein the antioxidant is a vitamin, provitamin, ascorbic acid, vitamin E or salts or esters thereof.

59. The composition of claim 54, wherein the stabilizer is ferric citrate, ferric phosphate or ferric pyrophosphate.

60. The composition of claim 1, further comprising a cosolvent.

61. The composition of claim 60, wherein the cosolvent is a hydroxylated solvent.

62. The composition of claim 60, wherein the cosolvent is an alcohol or a glycol.

63. The composition of claim 60, wherein the cosolvent is isopropyl alcohol, propylene glycol, polyethylene glycol, polypropylene glycol, glycol ether, glycerol, or a polyoxyethylene alcohol.

64. The composition of claim 60, wherein the cosolvent is a glycol.

65. The composition of claim 60, wherein the cosolvent is propylene glycol or polyethylene glycol

66. The composition of claim 60, wherein the cosolvent is polyethylene glycol 300 or 400.

67. The composition of claim 60, wherein the cosolvent is a glycol at a concentration of about 0% to about 50% by weight.

68. The composition of claim 60, wherein the cosolvent is a glycol at a concentration of about 1% to about 40% by weight.

69. The composition of claim 60, wherein the cosolvent is a glycol at a concentration of about 6% to about 20% by weight.

70. The composition of claim 60, wherein the cosolvent is propylene glycol.

71. The composition of claim 60, wherein the cosolvent is a glycol at a concentration of about 0% to about 15% by weight.

72. The composition of claim 60, wherein the cosolvent is a glycol at a concentration of about 0.1% to about 10% by weight.

73. The composition of claim 60, wherein the cosolvent is a glycol at a concentration of about 1% to about 4 or 5% by weight.

74. The composition of claim 1, further comprising an emulsifier.

75. The composition of claim 74, wherein the emulsifier is polyoxyetheylene sorbitan fatty esters or polysorbates, including, but not limited to, polyethylene sorbitan monooleate (Polysorbate 80), polysorbate 20 (polyoxyethylene (20) sorbitan monolaurate), polysorbate 65 (polyoxyethylene (20) sorbitan tristearate), polyoxyethylene (20) sorbitan mono-oleate, polyoxyethylene (20) sorbitan monopalmitate, polyoxyethylene (20) sorbitan monostearate; lecithins; alginic acid; sodium alginate; potassium alginate; ammonium alginate; calcium alginate; propane-1,2-diol alginate; agar; carrageenan; locust bean gum; guar gum; tragacanth; acacia; xanthan gum; karaya gum; pectin; amidated pectin; ammonium phosphatides; microcrystalline cellulose; methylcellulose; hydroxypropylcellulose; hydroxypropylmethylcellulose; ethylmethylcellulose; carboxymethylcellulose; sodium, potassium and calcium salts of fatty acids; mono- and di-glycerides of fatty acids; acetic acid esters of mono- and di-glycerides of fatty acids; lactic acid esters of mono- and di-glycerides of fatty acids; citric acid esters of mono- and di-glycerides of fatty acids; tartaric acid esters of mono- and di-glycerides of fatty acids; mono- and diacetyltartaric acid esters of mono- and di-glycerides of fatty acids; mixed acetic and tartaric acid esters of mono- and di-glycerides of fatty acids; sucrose esters of fatty acids; sucroglycerides; polyglycerol esters of fatty acids; polyglycerol esters of polycondensed fatty acids of castor oil; propane-1,2-diol esters of fatty acids; sodium stearoyl-2-lactylate; calcium stearoyl-2-lactylate; stearoyl tartrate; sorbitan monostearate; sorbitan tristearate; sorbitan monolaurate; sorbitan mono-oleate; sorbitan monopalmitate; extract of quillaia; polyglycerol esters of dimerised fatty acids of soya bean oil; oxidatively polymerised soya bean oil; or pectin extract.

76. The composition of claim 1, further comprising glycerin.

77. The composition of claim 76, comprising about 0% to about 50% by weight glycerin.

78. The composition of claim 76, comprising about 1% to about 40% by weight glycerin.

79. The composition of claim 76, comprising about 6% to about 20% by weight glycerin.

80. The composition of claim 76, comprising about 0% to about 15% by weight glycerin.

81. The composition of claim 76, comprising about 1% to about 10% by weight glycerin.

82. The composition of claim 76, comprising about 1% to about 5% by weight glycerin.

83. The composition of claim 1, comprising about 1% to about 69% by weight water.

84. The composition of claim 1, comprising about 15% to about 69% by weight water.

85. The composition of claim 1, comprising about 30% to about 69% by weight water.

86. The composition of claim 1, comprising about 5% to about 69% by weight water.

87. The composition of claim 1, comprising about 60% to about 69% by weight water.

88. The composition of claim 1, comprising about 40% to about 69% by weight water.

89. The composition of claim 1 that has been nebulized.

90. A kit, comprising (a) a composition of claim 1; and (b) a nebulizer.

91. An article of manufacture, comprising packaging material, a composition of claim 1, which is useful for treatment, prevention or amelioration of one or more symptoms of diseases or disorders associated with undesired and/or uncontrolled bronchoconstriction, and a label that indicates that the composition is used for treatment, prevention or amelioration of one or more symptoms of diseases or disorders associated with undesired and/or uncontrolled bronchoconstriction.

92. A method for treating, preventing, or ameliorating one or more symptoms of a bronchoconstrictive disorder, comprising administering a composition of claim 1 to a subject in need thereof.

93. The method of claim 92, wherein the bronchoconstrictive disorder is asthma, bronchial asthma, allergic asthma, intrinsic asthma, late asthma, airway hyper-responsiveness, or chronic bronchitis.

94. The method of claim 92, wherein the composition is administered via oral inhalation or nasally.

95. The method of claim 92, further comprising administering one or more of (a) or (b) as follows: (a) a β₂-adrenoreceptor agonist; or (b) a dopamine (D₂) receptor agonist; simultaneously with, prior to or subsequent to the composition provided herein.

96. The method of claim 95, wherein the _{β2}-adrenoreceptor agonists is Albuterol (α¹-(((1,1-dimethylethyl)amino)methyl)-4-hydroxy-1,3-benzenedimethanol); Bambuterol (dimethylcarbamic acid 5-(2-((1,1-dimethylethyl)amino)-1-hydroxyethyl)-1,3-phenylene ester); Bitolterol (4-methylbenzoic acid 4-(2-((1,1-dimethylethyl)amino)-1-hydroxyethyl)-1,2-phenylene ester); Broxaterol (3-bromo-α-(((1,1-dimethylethyl)amino)methyl)-5-isoxazolemethanol); Isoproterenol (4-(1-hydroxy-2-((1-methylethyl)amino)ethyl)-1,2-benzenediol); Trimetoquinol (1,2,3,4-tetrahydro-1-((3,4,5-trimethoxyphenyl)methyl)-6,7-isoquinolinediol); Clenbuterol (4-amino-3,5-dichloro-α-(((1,1-diemthylethyl)amino)methyl)benzenemethanol); Fenoterol (5-(1-hydroxy-2-((2-(4-hydroxyphenyl)-1-methylethyl)amino)ethyl)-1,3-benzenediol); Formoterol (2-hydroxy-5-((1RS)-1-hydroxy-2-(((1RS)-2-(p-methoxyphenyl)-1-methylethyl)amino)ethyl)formanilide); (R,R)-Formoterol; Desformoterol ((R,R) or (S,S)-3-amino-4-hydroxy-α-(((2-(4-methoxyphenyl)-1-methylethyl)amino)methyl)benzenemethanol); Hexoprenaline (4,4′-(1,6-hexanediyl)-bis(imino(1-hydroxy-2,1-ethanediyl)))bis-1,2-benzenediol); Isoetharine (4-(1-hydroxy-2-((1-methylethyl)amino)butyl)-1,2-benzenediol); Isoprenaline (4-(1-hydroxy-2-((1-methylethyl)amino)ethyl)-1,2-benzenediol); Metaproterenol (5-(1-hydroxy-2-((1-methylethyl)amino)ethyl)-1,3-benzenediol); Picumeterol (4-amino-3,5-dichloro-_{α}-(((6-(2-(2-pyridinyl) ethoxy)hexyl)amino)methyl)benzenemethanol); Pirbuterol (_{α}⁶-(((1,1-dimethylethyl)amino)methyl)-3-hydroxy-2,6-pyridinemethanol); Procaterol (((R*,S*)-(±)-8-hydroxy-5-(1-hydroxy-2-((1-methylethyl)amino)butyl)-2(1H)-quinolinone); Reproterol ((7-(3-((2-(3,5-dihydroxyphenyl)-2-hydroxyethyl)amino)propyl)-3,7-dihydro-1,3-dimethyl-1H-purine-2,6-dione); Rimiterol (4-(hydroxy-2-piperidinylmethyl)-1,2-benzenediol); Salbutamol ((±)-á¹-(((1,1-dimethylethyl)amino)methyl)-4-hydroxy-1,3-benzenedimethanol); (R)-Salbutamol; Salmeterol ((±)-4-hydroxy-α¹-(((6-(4-phenylbutoxy)hexyl)amino)methyl)-1,3-benzenedimethanol); (R)-Salmeterol; Terbutaline (5-(2-((1,1-dimethylethyl)amino)-1-hydroxyethyl)-1,3-benzenediol); Tulobuterol (2-chloro-α-(((1,1-dimethylethyl)amino)methyl)benzenemethanol); or TA-2005 (8-hydroxy-5-((1R)-1-hydroxy-2-(N-((1R)-2-(4-methoxyphenyl)-1-methylethyl)amino)ethyl)carbostyril hydrochloride); and the dopamine (D₂) receptor agonist is Apomorphine ((R)-5,6,6a,7-tetrahydro-6-methyl-4H-dibenzo[de,g]quinoline-10,11-diol); Bromocriptine ((5′α)-2-bromo-12′-hydroxy-2′-(1-methylethyl)-5′-(2-methylpropyl)ergotaman-3′,6′,18-trione); Cabergoline ((8â)-N-(3-(dimethylamino)propyl)-N-((ethylamino)carbonyl)-6-(2-propenyl)ergoline-8-carboxamide); Lisuride (N′-((8α)-9,10-didehydro-6-methylergolin-8-yl)-N,N-diethylurea), Pergolide ((8α)-8-((methylthio)methyl)-6-propylergoline); Levodopa (3-hydroxy-L-tryrosine); Pramipexole ((S)-4,5,6,7-tetrahydro-N⁶-propyl-2,6-benzothiazolediamine); Quinpirole hydrochloride (trans-(−)-4aR-4,4a,5,6,7,8,8a,9-octahydro-5-propyl-1H-pyrazolo[3,4-g]quinoline hydrochloride); Ropinirole (4-(2-(dipropylamino)ethyl)-1,3-dihydro-2H-indol-2-one); and Talipexole (5,6,7,8-tetrahydro-6-(2-propenyl)-4H-thiazolo[4,5-d]azepin-2-amine).

97. The method of claim 92, further comprising administering one or more of the following: an IL-5 inhibitor; an antisense modulators of IL-5; milrinone; milrinone lactate; a tryptase inhibitor; a tachykinin receptor antagonist; a leukotriene receptor antagonist; a 5-lypoxygenase inhibitor, or an anti-IgE antibody.

98. A process for preparing the composition of claim 1, comprising:
(a) dissolving the corticosteroid in the surfactant; (b) dissolving the alkali or alkaline earth metal halide in the water; (c) adding the corticosteroid/surfactant solution slowly to the alkali or alkaline earth metal halide/water solution.

99. The process of claim 98, further comprising adding a cosolvent to the surfactant prior to step (a).

100. The process of claim 98, wherein the corticosteroid/surfactant solution is maintained above about 45° C.

101. The process of claim 98, wherein the corticosteroid/surfactant solution is maintained above about 60° C.

102. The process of claim 98, wherein the sodium chloride/water solution is maintained at about ambient temperature.

103. The process of claim 98, further comprising: (d) filtering the solution resulting from step (c).

104. The process of claim 103, wherein the pore size of the filter is equal to or less than about 0.22 μm.

105. The process of claim 103, wherein the pore size of the filter is equal to or less than about 0.1 μm.

106. The process of claim 98, further comprising addition of one or more of (i) buffer, (ii) ferric citrate or (iii) glycerin to the aqueous phase prior to step (c).

107. A composition, comprising a corticosteroid anti-inflammatory agent and an alkali or alkaline earth metal halide, wherein the alkali or alkaline earth metal halide is present at a concentration of about 0.01% to about 0.89% by weight.

108. A combination, comprising a composition of claim 1 and a diluent.
